(19) United States
(12) Patent Application Publication
(10) Pub. No.: US 2006/0264809 A1
(43) Pub. Date: Nov. 23, 2006

(54) Ultrasound catheter with cavitation promoting surface

(76) Inventors: Douglas R. Hansmann, Bainbridge Island, WA (US); Azita Soltani, Snohomish, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/402,626

(22) Filed: Apr. 12, 2006

Related U.S. Application Data
(60) Provisional application No. 60/670,412, filed on Apr. 12, 2005.

(51) Int. Cl. A61B 17/20

(52) U.S. Cl. 604/22

(57) ABSTRACT

In one embodiment of the present invention, a method of applying ultrasonic energy to a treatment site within a patient's vasculature comprises positioning an ultrasound radiating member at a treatment site within a patient's vasculature. The method further comprises activating the ultrasound radiating member to produce pulses of ultrasonic energy at a cycle period T≦1 second. Each pulse of ultrasonic energy has a first peak amplitude for a first duration, and a second reduced amplitude that is less than the first peak amplitude for a second duration.

### PRIORITY APPLICATION

This application claims the benefit of U.S. Provisional Patent Application 60/670,412, filed 12 Apr. 2005, the entire disclosure of which is hereby incorporated by reference herein.

### CROSS-REFERENCE TO RELATED APPLICATIONS

This application is related to U.S. patent application Ser. No. 10/309,388 (filed 3 Dec. 2002; published as US 2004/0024347 A1; Attorney Docket EKOS.025A) and U.S. patent application Ser. No. 11/047,464 (filed 31 Jan. 2005; published as US 2005/0215942 A1; Attorney Docket EKOS.168A2). The entire disclosure of both of these related applications is hereby incorporated by reference herein.

### FIELD OF THE INVENTION

The present invention relates generally to ultrasound catheter systems, and more specifically to ultrasound catheter systems configured for the treatment of vascular occlusions.

### BACKGROUND OF THE INVENTION

Ultrasonic energy is often used to enhance the intravascular delivery and/or effect of various therapeutic compounds. Ultrasound catheters are used to deliver ultrasonic energy and therapeutic compounds to a treatment site within a patient's vasculature. Such ultrasound catheters typically comprise an elongate member configured to be advanced through a patient's vasculature and an ultrasound assembly that is positioned near a distal end portion of the elongate member. The ultrasound assembly is configured to emit ultrasonic energy. Ultrasound catheters often include a fluid delivery lumen that is used to deliver the therapeutic compound to the treatment site. In this manner, ultrasonic energy is delivered to the treatment site to enhance the effect and/or delivery of the therapeutic compound.

For example, ultrasound catheters have been successfully used to treat human blood vessels that have become occluded by plaque, thrombi, emboli or other substances that reduce the blood carrying capacity of the vessel. See, for example, U.S. Pat. No. 6,001,069. To remove the occlusion, the ultrasound catheter is advanced through the patient's vasculature to deliver a therapeutic compound containing dissolution compounds directly to the occlusion. To enhance the effect and/or delivery of the therapeutic compound, ultrasonic energy is emitted into the therapeutic compound and/or the surrounding tissue at the treatment site. In other applications, ultrasound catheters are used for other purposes, such as for the delivery and activation of light activated drugs. See, for example, U.S. Pat. No. 6,176,842.

### SUMMARY OF THE INVENTION

In some cases, introduction of excess ultrasonic energy to a treatment site within a patient's vasculature can cause unwanted heating of the treatment site. Thus, it is desired to operate the ultrasonic catheter in a way that does not produce such unwanted heating. One such method of operation involves reducing the average power delivered to the treatment site in each pulse of ultrasonic energy. Another such method of operation involves providing a cavitation promoting surface at the treatment site that enhances cavitation without the delivery of additional ultrasonic energy.

In one embodiment of the present invention, a method of applying ultrasonic energy to a treatment site within a patient's vasculature comprises positioning an ultrasound radiating member at a treatment site within a patient's vasculature. The method further comprises activating the ultrasound radiating member to produce pulses of ultrasonic energy at a cycle period T≦1 second. Each pulse of ultrasonic energy has a-first peak amplitude for a first duration, and a second reduced amplitude that is less than the first peak amplitude for a second duration.

In another embodiment of the present invention, a method comprises positioning an ultrasound radiating member at a treatment site within a patient's vasculature. The method further comprises delivering pulses of ultrasonic energy to the treatment site from the ultrasound radiating member. The pulses of ultrasonic energy include a variable amplitude, such that the pulses have an increased pulse amplitude during a first pulse segment, and a reduced pulse amplitude during a second pulse segment. The method further comprises delivering a therapeutic compound to the treatment site simultaneously with the delivery of the pulses of ultrasonic energy.

In another embodiment of the present invention, a method comprises positioning a catheter at a treatment site within a patient's vasculature. The catheter is positioned at least partially within an occlusion at the treatment site. The method further comprises delivering a therapeutic compound from the catheter to the occlusion. The method further comprises delivering a plurality of packets ultrasonic energy from an ultrasound radiating member positioned within the catheter to the occlusion. The packets of ultrasonic energy comprise a plurality of pulses of ultrasonic energy having an amplitude that varies pulse-to-pulse.

In another embodiment of the present invention, an ultrasound catheter is configured to be inserted into a patient's vascular system. The catheter comprises an elongate outer sheath defining a central lumen that extends longitudinally from an outer sheath proximal region to an outer sheath distal region. The catheter further comprises an elongate hollow inner core positioned in the central lumen. The inner core defines a utility lumen. The catheter further comprises a ultrasound radiating member having a hollow inner passage through which the inner core passes. The ultrasound radiating member is positioned generally between the inner core and the outer sheath. The outer sheath includes an outer surface. The outer sheath outer surface has a cavitation promoting region located adjacent to the ultrasound radiating member. The outer sheath outer surface also has a smooth region located proximal to the cavitation promotion region. The cavitation promoting region has an increased surface roughness as compared to the smooth region.

In another embodiment of the present invention, a catheter system for delivering ultrasonic energy and a therapeutic compound to a treatment site within a body lumen comprises a tubular body. The tubular body has a proximal end. The tubular body has a distal end. The tubular body has an energy delivery section positioned between the proximal end and the distal end. The energy delivery section includes a cavitation promoting surface having an increased surface roughness. The catheter system further comprises a fluid delivery lumen extending at least partially through the tubular body and having at least one outlet in the energy delivery section. The catheter system further comprises an inner core configured for insertion into the tubular body. The inner core comprises a plurality of ultrasound radiating members connected to an elongate electrical conductor. The catheter system further comprises wiring such that a voltage can be applied from the elongate electrical conductor across a selected plurality of the ultrasound radiating members. The selected plurality of ultrasound radiating members can be driven simultaneously.

In another embodiment of the present invention, A method of treating a vascular occlusion comprises delivering a catheter with a plurality of ultrasound radiating members to a treatment site within a patient's vasculature. The vascular occlusion is located at the treatment site. The catheter includes a cavitation promoting surface region having an increased surface roughness as compared to surface regions adjacent the cavitation promoting surface region. The method further comprises delivering ultrasonic energy to the treatment site from the catheter so as to generate cavitation at the treatment site.

In another embodiment of the present invention, an ultrasound catheter comprises an elongate tubular body having a proximal region and a distal region. An energy delivery section is included within the distal region of the tubular body. The ultrasound catheter further comprises an ultrasound radiating member positioned adjacent to the energy delivery section of the elongate tubular body. The ultrasound catheter further comprises a cavitation promoting surface that is formed on an exterior surface of the ultrasound catheter. The cavitation promoting surface is exposed to ultrasonic energy when the ultrasound radiating member is activated. The ultrasound catheter further comprises a fluid delivery lumen positioned within the elongate tubular body. The ultrasound catheter further comprises a fluid delivery port that is configured to deliver a fluid within the fluid delivery lumen to an exterior region of the ultrasound catheter that is adjacent to the cavitation promoting surface.

In another embodiment of the present invention, a catheter system comprises an elongate tubular body having a distal region and a proximal region opposite the distal region. The catheter system further comprises an ultrasound radiating member positioned adjacent to the distal region of the elongate tubular body. The catheter system further comprises a fluid delivery lumen extending through at least a portion of the elongate tubular body. The catheter system further comprises a fluid delivery port that is configured to deliver a fluid within the fluid delivery lumen to a region exterior to the elongate tubular body. The catheter system further comprises a control system configured to provide a control signal to the ultrasound radiating member. The control signal causes the ultrasound radiating member to generate a plurality of pulses of ultrasonic energy. A first pulse of ultrasonic energy has an amplitude that is greater than a second pulse of ultrasonic energy.

In another embodiment of the present invention, a catheter system comprises an elongate tubular body having a distal region and a proximal region opposite the distal region. The catheter system further comprises an ultrasound radiating member positioned adjacent to the distal region of the elongate tubular body. The catheter system further comprises a fluid delivery lumen extending through at least a portion of the elongate tubular body. The catheter system further comprises a fluid delivery port that is configured to deliver a fluid within the fluid delivery lumen to a region exterior to the elongate tubular body. The catheter system further comprises a control system configured to provide a control signal to the ultrasound radiating member. The control signal causes the ultrasound radiating member to generate pulses of ultrasonic energy at a cycle period T≦1 second. A selected pulse of ultrasonic energy has a first peak amplitude for a first duration, and a second reduced amplitude that is less than the first peak amplitude for a second duration.

### BRIEF DESCRIPTION OF THE DRAWINGS

Exemplary embodiments of the cavitation promoting systems and methods disclosed herein are illustrated in the accompanying drawings, which are for illustrative purposes only. The drawings comprise the following figures, in which like numerals indicate like parts.

FIG. 1A is a schematic illustration of a stable microbubble located within a crevice of a roughened surface.

FIG. 1B is a schematic illustration of the expansion of the stable microbubble of FIG. 1A, which occurs upon exposure to the rarefaction portion of an acoustic wave.

FIG. 1C is a schematic illustration of a free microbubble expelled from the crevice of FIG. 1A.

FIG. 2A is an axial cross-sectional view of selected components of an exemplary ultrasound catheter assembly that is particularly well-suited for treatment of peripheral vascular occlusions, and that includes a cavitation promoting surface.

FIG. 2B is a longitudinal cross-sectional view of selected components of an exemplary ultrasound catheter assembly that is particularly well-suited for treatment of cerebral vascular occlusions, and that includes a cavitation promoting surface.

FIG. 3 is a plot of relative lysis of an in vitro plasma clot model as a function of ultrasonic energy exposure time for selected example embodiments.

FIG. 4 is a plot of average broadband noise detected as a function of peak acoustic pressure of ultrasonic energy exposed to various cavitation promoting surfaces.

FIG. 5A is a sonogram illustrating microbubble activity around a cavitation promoting surface in a plasma clot without the addition of a therapeutic compound.

FIG. 5B is a sonogram illustrating microbubble activity around a cavitation promoting surface in a plasma clot when a therapeutic compound is added to the treatment site.

FIG. 6A is a microscopic image (200×) of a plain polyimide surface.

FIG. 6B is a microscopic image (200×) of a polyimide surface having polytetrafluoroethylene particles dispersed therein.

FIG. 7 schematically illustrates an example ultrasonic energy pulse profile.

FIG. 8 illustrates an ultrasonic waveform having an elevated average pulse power.

FIG. 9 illustrates a modified ultrasonic waveform having a reduced average pulse power.

FIG. 10 illustrates a second modified ultrasonic waveform having a reduced average pulse power.

FIG. 11 illustrates a third modified ultrasonic waveform having a reduced average pulse power.

FIG. 12 illustrates a modified ultrasonic waveform having a gradually increasing pulse power.

FIG. 13 illustrates a modified ultrasonic waveform having a plurality of smaller pulses of ultrasonic energy.

FIG. 14 illustrates a modified ultrasonic waveform having a plurality of pulses having a sinusoidally-varying peak amplitude.

FIG. 15 illustrates a modified ultrasonic waveform having a plurality of pulses delivered in an envelope that is followed by a period of little or no delivery of ultrasonic energy.

FIG. 16 is a schematic illustration of certain features of an example ultrasonic catheter.

FIG. 17 is a block diagram of an example feedback control system for use with an ultrasound catheter.

### DETAILED DESCRIPTION OF THE INVENTION

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHz and about 3 MHz in another example embodiment, of about 3 MHz in another example embodiment, and of about 2 MHz in another example embodiment. As used herein, the term “catheter” is used broadly, includes its ordinary meaning, and further includes an elongate flexible tube configured to be inserted into the body of a patient, such as into a body cavity, duct or vessel. As used herein, the term “therapeutic compound” is used broadly, includes its ordinary meaning, and encompasses drugs, medicaments, dissolution compounds, genetic materials, and other substances capable of effecting physiological functions. A mixture comprising such substances is encompassed within this definition of “therapeutic compound”. As used herein, the term “end” is used broadly, includes its ordinary meaning, and further encompasses a region generally, such that “proximal end” includes “proximal region”, and “distal end” includes “distal region”.

As expounded herein, ultrasonic energy is often used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be particularly advantageous in the context of thrombolytic therapy, chemotherapy, radiation therapy, and gene therapy, as well as in applications calling for the delivery of proteins and/or therapeutic humanized antibodies.

The beneficial effect of ultrasonic energy described herein has been found to be enhanced in the presence of cavitation. As used herein, the term “cavitation” is used broadly, includes its ordinary meaning, and further refers to the formation and/or driven vibration of bubbles in liquids by sonically induced mechanical forces of ultrasonic energy. Under certain conditions, these bubbles are made to form, grow, and collapse in less than one microsecond, resulting in the creation of bursts of intense and highly localized energy. This phenomenon is referred to as “inertial cavitation”. Under other conditions, these bubbles are made to oscillate in a steady state fashion, resulting in the creation of small scale fluid flows called micro-streaming. This phenomenon is referred to as “stable cavitation”. Inertial cavitation has the potential to create transitory free radicals via molecular dissociation, and launch high velocity liquid micro-jets.

Stable cavitation and inertial cavitation have acoustic signatures that are usable to distinguish these phenomena from each other. Specifically, subharmonic and ultra-harmonic noise are indicators of stable cavitation, while broadband noise is an indicator of inertial cavitation. The frequencies that are considered to be subharmonic and ultra-harmonic are determined based on the harmonic frequency of the ultrasound radiating member used to generate the ultrasonic energy.

The acoustic parameters of the ultrasonic energy influence cavitation inception. Such parameters include pressure amplitude, frequency, duty cycle and pulse duration. FIG. 7 schematically illustrates an example ultrasonic energy pulse profile 100 having a first pressure amplitude 102 and a second pressure amplitude 104. In other embodiments, the pulse profile includes a constant pressure amplitude, or a variable pressure amplitude. Therefore, the pressure amplitude is often expressed as both a peak acoustic pressure and an average acoustic pressure. The pulse profile 100 illustrated in FIG. 7 has a pulse duration 106, during which a plurality of burst cycles 108 occur. Often the pulse duration is expressed as a number of burst cycles that occur during the pulse. Additional information regarding ultrasonic energy pulse profiles is provided in U.S. Provisional Patent Application 60/670,412 (filed 12 Apr. 2005), the entire disclosure of which is hereby incorporated by reference herein.

In an example embodiment, cavitation is generated at an intravascular treatment site using ultrasonic energy having a pressure amplitude greater than about 1 MPa. In an example embodiment, cavitation is generated at an intravascular treatment site using ultrasonic energy having a frequency that is preferably between about 1 MHz and about 3 MHz, and more preferably between about 1.7 MHz and about 2.2 MHz. In an example embodiment, cavitation is generated at an intravascular treatment site using ultrasonic energy having a duty cycle between about 0.001% and about 50%. In an example embodiment, inertial cavitation is generated at an intravascular treatment site using ultrasonic energy having a pulse duration between that is preferably between about 1 burst cycle and about 7000 burst cycles, and that is more preferably between about 10 burst cycles and 1000 burst cycles.

The threshold acoustic pressure amplitude to initiate, and optionally sustain, cavitation at least partially depends on both duty cycle and pulse duration. For instance, depending on the dissolved gas content of the blood surrounding the catheter, the threshold pressure amplitude for a 1-cycle pulse of ultrasonic energy is different than the threshold pressure amplitude to a 50-cycle pulse of ultrasonic energy. The risk of causing thermal damage to the treatment site and/or reducing ultrasound radiating member lifetime is mitigated by avoiding long duty cycles and/or high pressure amplitudes, or by otherwise adjusting the acoustic parameters of the ultrasonic energy.

Disclosed herein are methods for enhancing the beneficial defect of ultrasonic energy at an intravascular treatment site by promoting cavitation at the treatment site. Aside from manipulating the acoustic parameters of the ultrasonic energy, other techniques for promoting cavitation at the treatment site include supplying an ultrasound contrast agent to the treatment site and/or using an ultrasound catheter that includes a cavitation promoting surface. Use of such techniques reduces the acoustic pressure amplitude required to initiate cavitation, and therefore allows lower levels of ultrasonic energy to be delivered to the treatment site from the ultrasound assembly. This provides several advantages, such as prolonging the life of a ultrasound radiating member and reducing the likelihood of causing thermal damage to the treatment site. While cavitation is used to enhance the delivery and/or effect of a therapeutic compound in certain embodiments, cavitation promotes clot dissolution even in the absence of a therapeutic compound. Indeed, in the context of treating a vascular occlusion, the beneficial effect of cavitation in the absence of a therapeutic compound is often greater than the beneficial effect of a therapeutic compound alone.

Because cavitation promoting surfaces and ultrasound contrast agents are independently capable of inducing cavitation at an intravascular treatment site, in certain embodiments cavitation is induced at an intravascular treatment site using a cavitation promoting surface, but without using an ultrasound contrast agent. Such embodiments advantageously simplify the treatment procedure by eliminating the need to monitor the concentration of the ultrasound contrast agent at the treatment site, reduce the treatment cost, and reduce the risk of systemic complications caused by the ultrasound contrast agent. In other embodiments, cavitation is induced at an intravascular treatment site using a ultrasound contrast agent, but without using a cavitation promoting surface. Such embodiments advantageously are usable with conventional ultrasound catheters that have not been modified to include the cavitation promoting surface. In still other embodiments, both a cavitation promoting surface and an ultrasound contrast agent are used to enhance cavitation at the treatment site. Regardless of whether a ultrasound contrast agent, a cavitation promoting surface, or both, are used to promote cavitation, the generation of free microbubbles at the treatment site is optionally manipulated by adjusting the frequency, peak pressure and duration of ultrasonic energy delivered to the treatment site.

The techniques disclosed herein are compatible with a wide variety of ultrasound catheters, several examples of which are disclosed in USA Patent Application Publication US 2004/0024347 A1 (published 5 Feb. 2004; discloses catheters especially well-suited for use in the peripheral vasculature) and USA Patent Application Publication 2005/0215942 A1 (published 29 Sep. 2005; discloses catheters especially well-suited for use in the cerebral vasculature). Certain of the techniques disclosed herein are compatible with ultrasound catheters that would be unable to generate cavitation at an intravascular treatment site but for the use of such techniques.

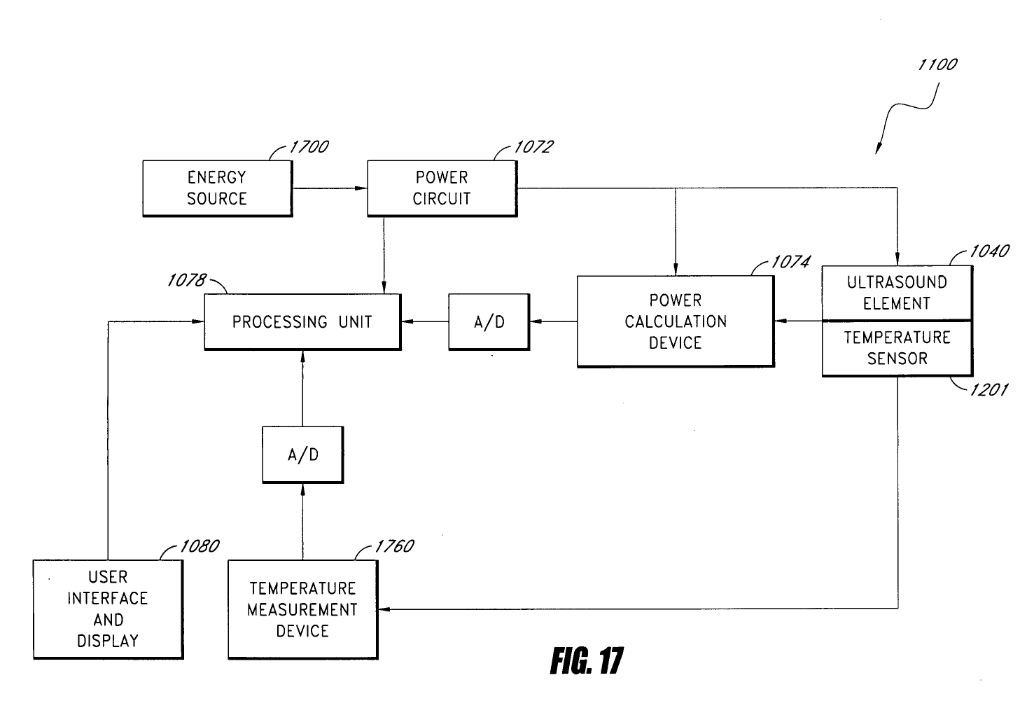

FIG. 16 illustrates an ultrasonic catheter 1000 configured for use in a patient's vasculature. For example, in certain applications the ultrasonic catheter 1000 is used to treat long segment peripheral arterial occlusions, such as those in the vascular system of the leg, while in other applications the ultrasonic catheter 1000 is used to treat occlusions in the small vessels of the neurovasculature. Thus, the dimensions of the catheter 1000 are adjusted based on the particular application for which the catheter 1000 is to be used.

The ultrasonic catheter 1000 generally comprises a multi-component, elongate flexible tubular body 1200 having a proximal region 1400 and a distal region 1500. The tubular body 1200 includes a flexible energy delivery section 1800 located in the distal region 1500 of the catheter 1000. The tubular body 1200 and other components of the catheter 1000 are manufactured in accordance with a variety of techniques. Suitable materials and dimensions are selected based on the natural and anatomical dimensions of the treatment site and on the desired percutaneous access site.

For example, in a preferred embodiment the proximal region 1400 of the tubular body 1200 comprises a material that has sufficient flexibility, kink resistance, rigidity and structural support to push the energy delivery section 1800 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 1400 of the tubular body 1200 is reinforced by braiding, mesh or other constructions to provide increased kink resistance and pushability. For example, in certain embodiments nickel titanium or stainless steel wires are placed along or incorporated into the tubular body 1200 to reduce kinking.

The energy delivery section 1800 of the tubular body 1200 optionally comprises a material that (a) is thinner than the material comprising the proximal region 1400 of the tubular body 1200, or (b) has a greater acoustic transparency than the material comprising the proximal region 1400 of the tubular body 1200. Thinner materials generally have greater acoustic transparency than thicker materials. Suitable materials for the energy delivery section 1800 include, but are not limited to, high or low density polyethylenes, urethanes, nylons, and the like. In certain modified embodiments, the energy delivery section 1800 is formed from the same material or a material of the same thickness as the proximal region 1800.

One or more fluid delivery lumens are incorporated into the tubular body 1200. For example, in one embodiment a central lumen passes through the tubular body 1200. The central lumen extends through the length of the tubular body 1200, and is coupled to a distal exit port 1290 and a proximal access port 1310. The proximal access port 1310-forms part of the backend hub 1330, which is attached to the proximal region 1400 of the catheter 1000. The backend hub 1330 optionally further comprises cooling fluid fitting 1460, which is hydraulically connected to a lumen within the tubular body 1200. The backend hub 1330 also optionally comprises a therapeutic compound inlet port 1320, which is hydraulically connected to a lumen within the tubular body 1200. The therapeutic compound inlet port 1320 is optionally also hydraulically coupled to a source of therapeutic compound via a hub such as a Luer fitting.

The catheter 1000 is configured to have one or more ultrasound radiating members positioned therein. For example, in certain embodiments an ultrasound radiating member is fixed within the energy delivery section 1800 of the tubular body, while in other embodiments a plurality of ultrasound radiating members are fixed to an assembly that is passed into the central lumen. In either case, the one or more ultrasound radiating members are electrically coupled to a control system 1100 via cable 1450.

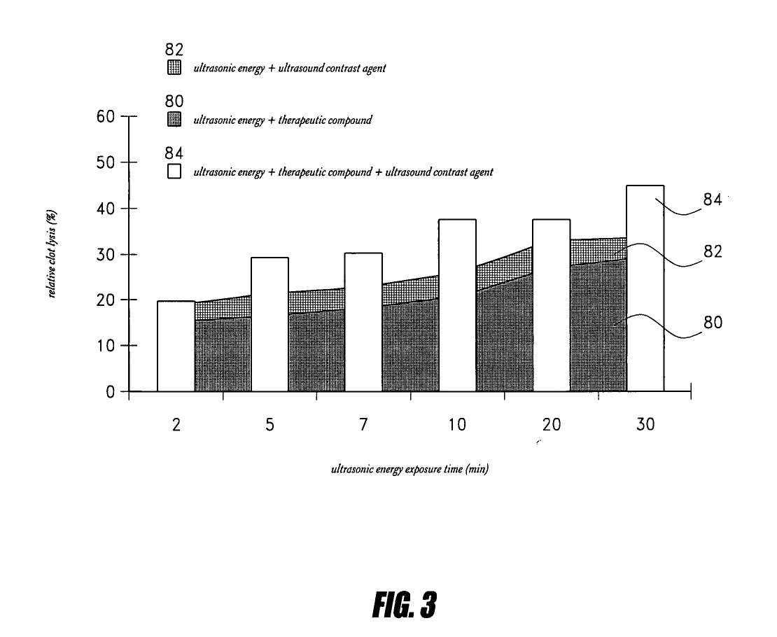

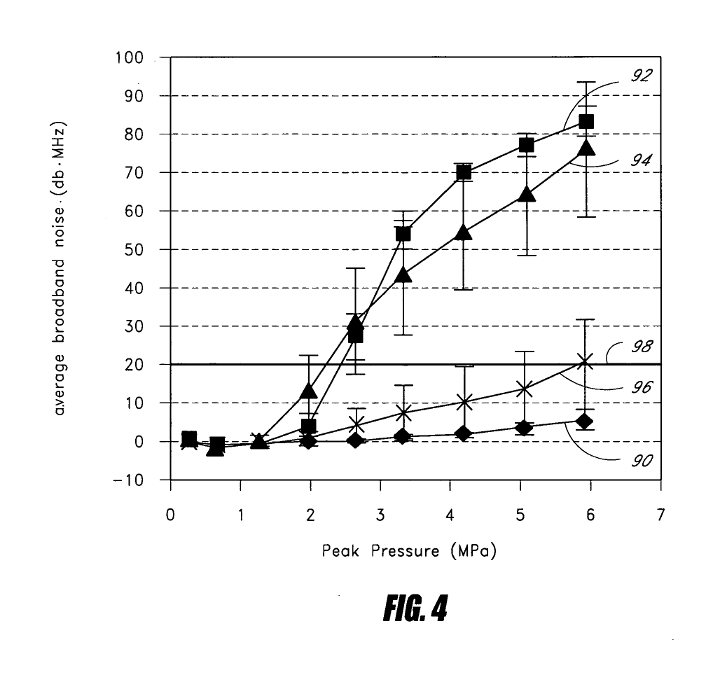

FIG. 2A illustrates an axial cross-sectional view of selected components of an exemplary ultrasound catheter assembly 60 that is particularly well-suited for treatment of peripheral vascular occlusions, and that includes a cavitation promoting surface 61. The catheter assembly 60 includes a therapeutic compound delivery lumen 62, a cooling fluid delivery lumen 63, a temperature sensor 64, and an ultrasound core 65 capable of housing an ultrasound radiating member array 66. Certain of these components are optional, and are omitted from alternative embodiments. The location of the cavitation promoting surface 61 on the catheter assembly 60 is selected based on the location of the ultrasound radiating member array 66. In an example embodiment, the cavitation promoting surface 61 is disposed only over regions of the catheter body 67 that are adjacent to regions where the ultrasound radiating member array 66 is configured to be positioned. So limiting the spatial extent of the cavitation promoting surface 61 advantageously causes the cavitation promoting surface 61 to have a reduced adverse effect, if any, on the intravascular maneuverability of the catheter assembly 60. In an example embodiment, the outer diameter of the catheter body 67 is approximately 0.043 inches, although other dimensions are used in other embodiments.

Similarly, FIG. 2B illustrates a longitudinal cross-sectional view of selected components of an exemplary ultrasound catheter assembly 70 that is particularly well-suited for treatment of cerebral vascular occlusions, and that includes a cavitation promoting surface 71. In the illustrated embodiment, the cavitation promoting surface 71 is formed on a ultrasound radiating member sheath 75, although in modified embodiments wherein the sheath 75 is omitted, the cavitation promoting surface 71 is formed directly on the catheter outer body 76. The catheter assembly 70 includes an inner core 73 that defines a utility lumen 72 configured to pass materials such as a guidewire, a therapeutic compound and/or a cooling fluid. The catheter assembly 70 further includes a distal tip element 74 and a hollow cylindrical ultrasound radiating member 77 that is mounted on the inner core 73. Certain of these components are optional, and are omitted from alternative embodiments. In an example embodiment, the cavitation promoting surface 71 is only positioned adjacent to the ultrasound radiating member 77. So limiting the spatial extent of the cavitation promoting surface 71 advantageously causes the cavitation promoting surface 71 to have a reduced adverse effect, if any, on the intravascular maneuverability of the catheter assembly 70. In an example embodiment, the diameter of the catheter outer body 76 is less than about 5 French, although other dimensions are used in other embodiments.

In example embodiments, the ultrasound radiating member 77 illustrated in FIG. 2B is a tubular piezoceramic transducer that is able to radiate ultrasonic energy in a length mode, a thickness mode, and a circumferential mode. The ultrasound radiating member 77 is capable of generating a pulse average spatial peak power this is preferably between about 78 W cm⁻² and about 98 W cm⁻², and is more preferably about 88 W cm⁻² . This results in the generation of peak acoustic pressures that are preferably between about 0.7 MPa and about 2.2 MPa, and that are more preferably between about 1.2 MPa and about 1.6 MPa.

In a modified embodiment, the ultrasound radiating member 77 has a resonant frequency greater than or equal to approximately 1 MHz in the thickness mode. In certain embodiments, the ultrasound radiating member included in an ultrasound catheter optionally includes an electrode, such as a nickel-plated electrode, that enables electrical wires to be soldered thereto.

FIG. 17 illustrates one embodiment of a feedback control system 1100 that is usable with certain of the embodiments disclosed herein, and that is illustrated in FIG. 16. The feedback control system 1100 allows the temperature at a temperature sensor 1201 to be monitored and allows the output power of an energy source 1700 to be adjusted accordingly. A physician is optionally able to override the closed or open loop system. The feedback control system 1100 includes the energy source 1700, a power circuit 1072 and a power calculation device 1074 that is coupled to an ultrasound radiating members 1040. A temperature measurement device 1760 is coupled to the temperature sensor 1201, which is positioned in the tubular body 1200. A processing unit 1078 is coupled to the power calculation device 1074, the power circuits 1072 and a user interface and display 1080.

In operation, the temperature at the temperature sensor 1201 is determined by the temperature measurement device 1760. The processing unit 1078 receives each determined temperature from the temperature measurement device 1760. The determined temperature can then be displayed to the user at the user interface and display 1080. The user interface and display 1080 is capable of receiving user input, such as a user-defined desired temperature. In a modified embodiment, the desired temperature is preset within the processing unit 1078, and is not user-modifiable. The processing unit 1078 comprises logic for generating a temperature control signal. The temperature control signal is proportional to the difference between the measured temperature and a desired temperature.

The temperature control signal is received by the power circuits 1072. The power circuits 1072 are optionally configured to adjust the power level, voltage, phase and/or current of the electrical energy supplied to the ultrasound radiating member 1040 from the energy source 1700. For example, when the temperature control signal is above a particular level, the power supplied to the ultrasound radiating member 1040 is reduced in response to that temperature control signal. Similarly, when the temperature control signal is below a particular level, the power supplied to the ultrasound radiating member 1040 is increased in response to that temperature control signal. After each power adjustment, the processing unit 1078 optionally monitors the temperature sensors 1201 and produces another temperature control signal which is received by the power circuits 1072.

Optionally the processing unit 1078 further comprises safety control logic. For example, it is generally desirable to prevent tissue at a treatment site from increasing more than 6° C. The safety control logic detects when the temperature at a temperature sensor 1201 has exceeded a safety threshold. The processing unit 1078 then generates a temperature control signal which causes the power circuits 1072 to stop the delivery of energy from the energy source 1700 to the ultrasound radiating member 1040. In other embodiments, the output from the power circuit 1072 maintains a selected energy for the ultrasound radiating member 1040 for a selected length of time.

In certain embodiments, the processing unit 1078 also receives a power signal from a power calculation device 1074. The power signal is used to determine the power being received by the ultrasound radiating member 1040. The determined power is then displayed to the user on the user interface and display 1080.

The processing unit 1078 can comprise a digital or analog controller, such as a computer with software. In embodiments wherein the processing unit 1078 is a computer, it optionally includes a central processing unit (“CPU”) coupled through a system bus. The user interface and display 1080 optionally comprises a mouse, a keyboard, a disk drive, a display monitor, and a nonvolatile memory system. Also optionally coupled to the bus is a program memory and a data memory.

In lieu of the series of power adjustments described above, a profile of the power to be delivered to the ultrasound radiating member 1040 is incorporated into the processing unit 1078, such that a preset amount of ultrasonic energy to be delivered is pre-profiled. In such embodiments, the power delivered to the ultrasound radiating member 1040 is then adjusted according to the preset profiles. For example, disclosed herein are a plurality of ultrasound waveforms which are optionally incorporated into the processing unit 1078. The processing unit is also optionally capable of independently controlling a plurality of ultrasound radiating members, either on an individual basis or on a grouped basis.

As used herein, the term “ultrasound contrast agent” is used broadly, includes its ordinary meaning, and further refers to a compound containing stabilized gas-filled nano-bubbles and microbubbles having a diameter in the range of about 10 nm to about 50 μm. While ultrasound contrast agents are commonly used with ultrasound imaging systems for diagnostic purposes, they also act as exogenous sources of cavitation nuclei. Acoustically activated ultrasound contrast agents have been shown to enhance thrombolysis and to enhance therapeutic compound delivery. Systemic delivery of an ultrasound contrast agent to an intravascular treatment site is relatively inefficient and carries the risk of systemic complications caused by high dosage levels. Therefore, local delivery of the ultrasound contrast agent directly to the treatment site using an ultrasound catheter capable of providing fluid delivery is generally preferred.

FIG. 3 is a plot of relative lysis of an in vitro plasma clot model as a function of ultrasonic energy exposure time for selected example embodiments. The ultrasonic energy used to obtain the data illustrated in FIG. 3 had a frequency of about 1 MHz, a peak pressure of about 1.6 MPa, and a duty cycle of about 7.5%. In a first example embodiment, a plasma clot model was exposed to ultrasonic energy and a therapeutic compound. In a second example embodiment, a plasma clot model was exposed to ultrasonic energy and an ultrasound contrast agent. In a third example embodiment, a plasma clot model was exposed to ultrasonic energy, a therapeutic compound, and an ultrasound contrast agent. In these three example embodiments, the therapeutic compound was ACTIVASE® tissue plasminogen activator (available from Genentech, Inc. (South San Francisco, Calif.)), and the ultrasound contrast agent was OPTISON® (available from Mallinckrodt Pharmaceuticals (Saint Louis, Mo.)). The lysis of the plasma clot model for these three example embodiments was compared to the lysis of a plasma clot model treated with a therapeutic compound only.

In FIG. 3, shaded region 80 indicates the relative lysis of the plasma clot model treated with ultrasonic energy and a therapeutic compound, shaded region 82 indicates the relative lysis of the plasma clot model treated with ultrasonic energy and an ultrasound contrast agent, and shaded region 84 indicates the relative lysis of the plasma clot model treated with ultrasonic energy, a therapeutic compound and an ultrasound contrast agent. The data presented in FIG. 3 indicates that the combination of the ultrasound contrast agent and the therapeutic compound produces a synergistic clot lysis effect, rather than a purely additive one. Specifically, once the ultrasonic energy exposure time is at least about five minutes, the relative clot lysis for a treatment that combines a therapeutic compound and an ultrasound contrast agent is significantly greater than the sum of the relative clot lysis for individual treatments that use only a therapeutic compound or only a ultrasound contrast agent.

When hydrophobic materials with or without a roughened surface texture are immersed in a liquid, small gas pockets are held in the small cracks and crevices of the roughened surface. Such immersion is often referred to as “imperfect wetting”. The gas pockets are stabilized against dissolution in the immersion liquid. Examples of such surfaces include roughened polytetrafluoroethylene surfaces and roughened polyimide surfaces. Like the microbubbles in an ultrasound contrast agent, these gas pockets are also able to act as a source of cavitation nuclei. Specifically, in certain embodiments ultrasonic energy is used to extract bubbles from the stabilized gas pockets on a roughened hydrophobic surface; the extracted free microbubbles are then used as a source of cavitation nuclei. Such a surface is typically referred to as a cavitation promoting surface. As described herein, and as illustrated in FIGS. 2A and 2B, cavitation promoting surfaces are incorporated onto an exterior surface of certain embodiments of an intravascular catheter.

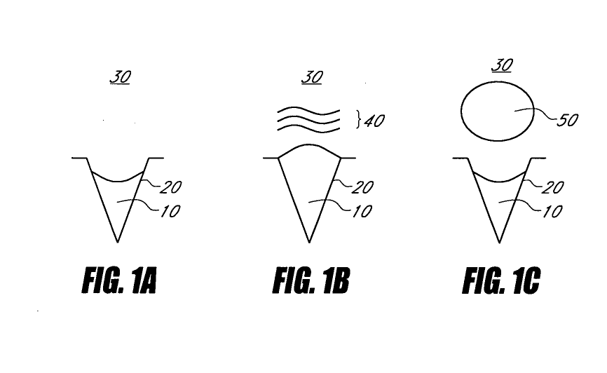

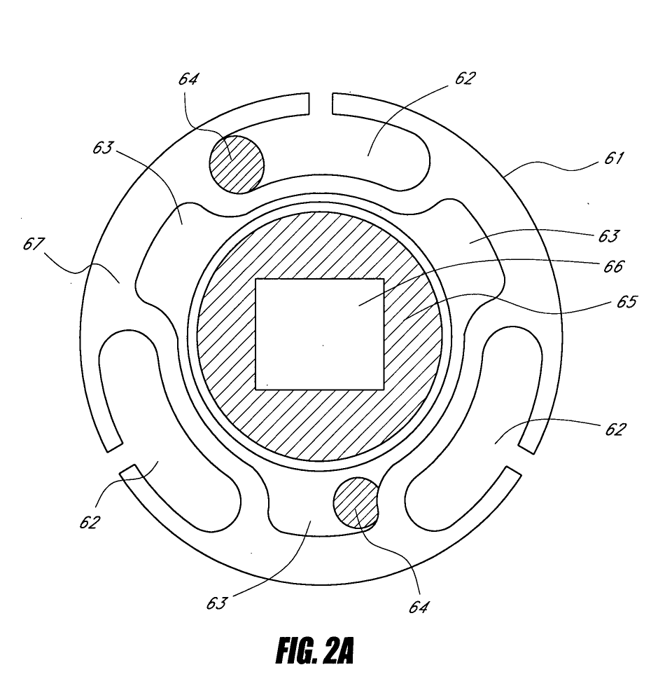

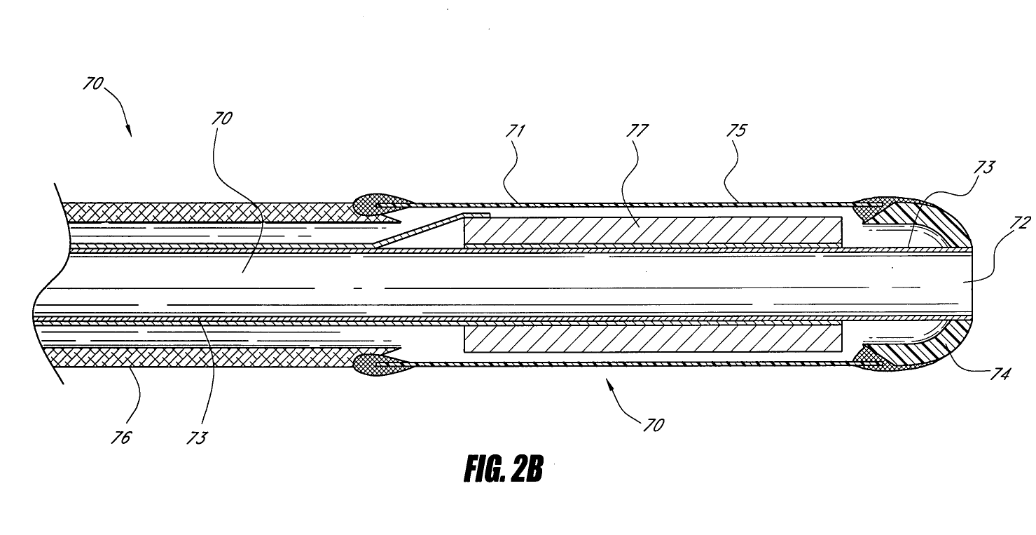

The phenomenon of cavitation nucleation on a cavitation promoting surface is similar in some respects to the phenomenon of boiling in that the threshold for bubble formation depends on the presence and interfacial tension of stabilized gas pockets on a roughened surface. FIG. 1A illustrates a stable gas pocket 10 located within a crevice 20 that is surrounded by a liquid 30. As illustrated in FIG. 1B, when the stable gas pocket 10 is exposed to the rarefaction portion of an acoustic wave 40, the volume of the stable gas pocket increases in response to the reduced pressure in the surrounding liquid 30. As illustrated in FIG. 1C, a portion of the stable gas pocket 10 is pinched off and expelled from the crevice 20, thereby forming a free microbubble 50. In this example, the crevice 20 acts as a cavitation nucleation site that is “activated” when exposed to ultrasonic energy having sufficient oscillating mechanical pressure to expel free microbubbles.

Thus, similar to the way that adding stones, chips or granules to a liquid lowers the boiling temperature of the liquid, adding a roughened surface to a catheter lowers the acoustic pressure threshold required to obtain ultrasonic cavitation over the catheter surface. This is particularly advantageous in the context of inducing cavitation at a treatment site using an ultrasound catheter, since the threshold pulse average spatial peak power intensity for generating free bubbles in the absence of a cavitation promoting surface (that is, from a smooth catheter surface) is as high as 19000 W cm⁻² when using a 1.8 MHz focused ultrasound field with an exposure duration of between 12 ms and 250 ms. The threshold acoustic pressure for inducing cavitation in the absence of a cavitation promoting surface is greater than 6.3 MPa, but is as low as about 2.7 MPa in the presence of a cavitation promoting surface. Thus, use of a cavitation promoting surface reduces the quantity of ultrasonic energy that must be delivered to the treatment site to induce cavitation, thereby advantageously (a) extending the operating lifetime of the ultrasound radiating members used to deliver the ultrasonic energy, and (b) increasing the safety of the treatment by decreasing the likelihood of causing damage to the treatment site.

Because liquids tend not to uniformly wet hydrophobic materials, such materials are generally well-suited for providing a high density of cavitation nucleation sites. Modifying the surface of such materials, such as by roughening the surface to produce additional cracks and crevices, causes even more cavitation nucleation sites to be created. For a surface with relatively small crevices (dimension less than or equal to about 10 μm), the surface tension is a dominating influential factor for microbubble nucleation.

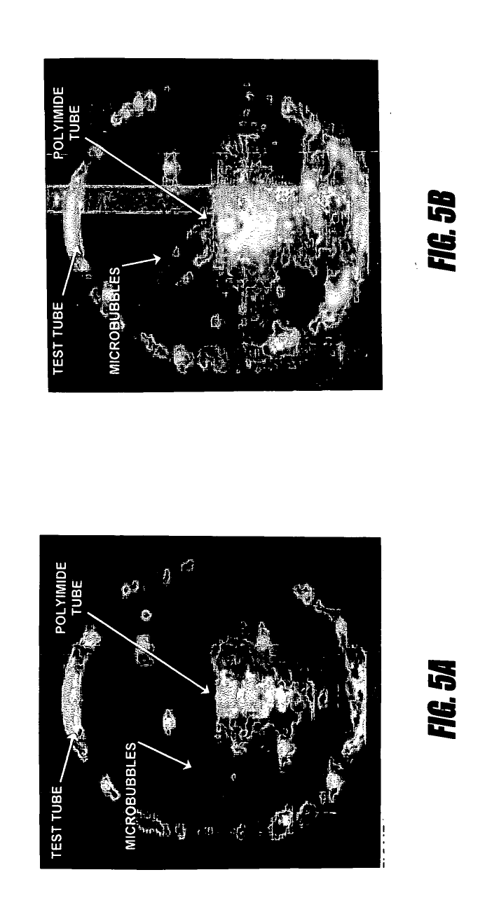

In certain applications, the efficacy of a particular catheter surface in promoting cavitation is determined by immersing the surface in a representative fluid (such as filtered gas-saturated water at 37° C. or plasma clot at 37° C.), exposing the surface to ultrasonic energy, and observing the amount of microbubble activity that is generated. For example, in one application a catheter surface is exposed to ultrasonic energy and the average broadband noise is determined as a function of peak acoustic pressure generated by the ultrasonic energy. FIG. 4 illustrates the results of such a determination for a smooth polyimide surface (line 90), a sanded polyimide surface (line 92), a surface with a polytetrafluoroethylene coating (line 94), and a surface with a parylene coating (line 96). Polytetrafluoroethylene coatings and parylene coatings are both hydrophobic, although parylene has a much finer surface roughness than polytetrafluoroethylene.

Inertial cavitation is indicated where the average broadband noise for a particular catheter surface is greater than the broadband noise detection threshold for a particular detection apparatus, as indicated by line 98. In an example embodiment, the broadband noise detection threshold is based on the broadband noise observed for a catheter without a cavitation promoting surface in a medium with a high cavitation threshold exposed to ultrasonic energy with a low pressure amplitude. FIG. 4 indicates that polytetrafluoroethylene coatings and sanded polyimide coatings serve as particularly effective cavitation promoting surfaces in certain embodiments, as these surfaces have particularly low acoustic pressure thresholds for producing steady inertial cavitation.

Stable cavitation is indicated where the magnitude of subharmonic noise for a particular catheter surface is greater than the subharmonic noise detection threshold for a particular detection apparatus. The magnitude of subharmonic noise for a particular catheter surface is obtained by first performing a fast Fourier transform (“FFT”) of the measured time domain signals, and then determining the amplitude of the FFT spectrum at half of the fundamental frequency (that is, the subharmonic frequency) of the ultrasound radiating member. The local noise floor around the subharmonic frequency is optionally subtracted from this amplitude to account for subharmonic signals due to elevated broadband noise levels caused by inertial cavitation. In an example embodiment, the subharmonic noise detection threshold is based on the subharmonic noise observed for a catheter without a cavitation promoting surface in a medium with a high cavitation threshold exposed to ultrasonic energy with a low pressure amplitude. The aggregate extent of cavitation activity can be quantified by integrating the detected noise over the duration of the treatment.

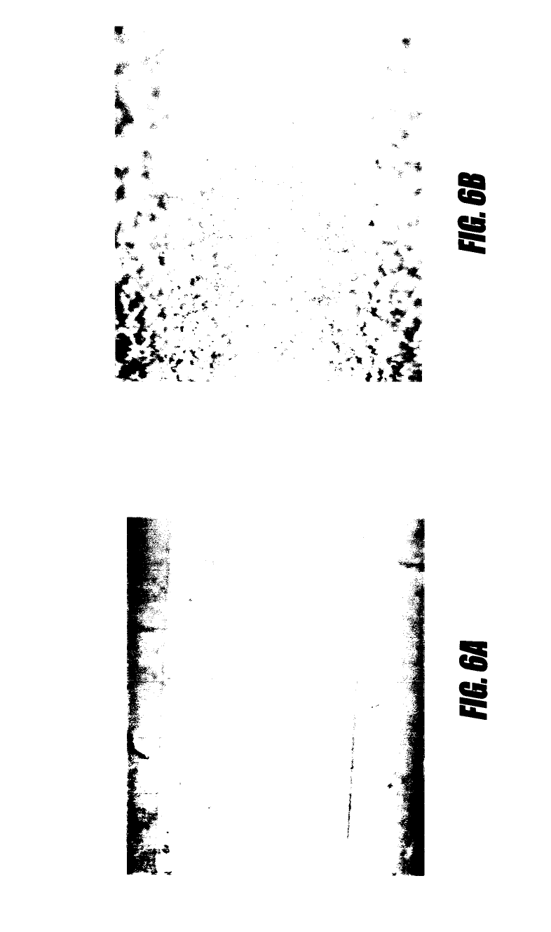

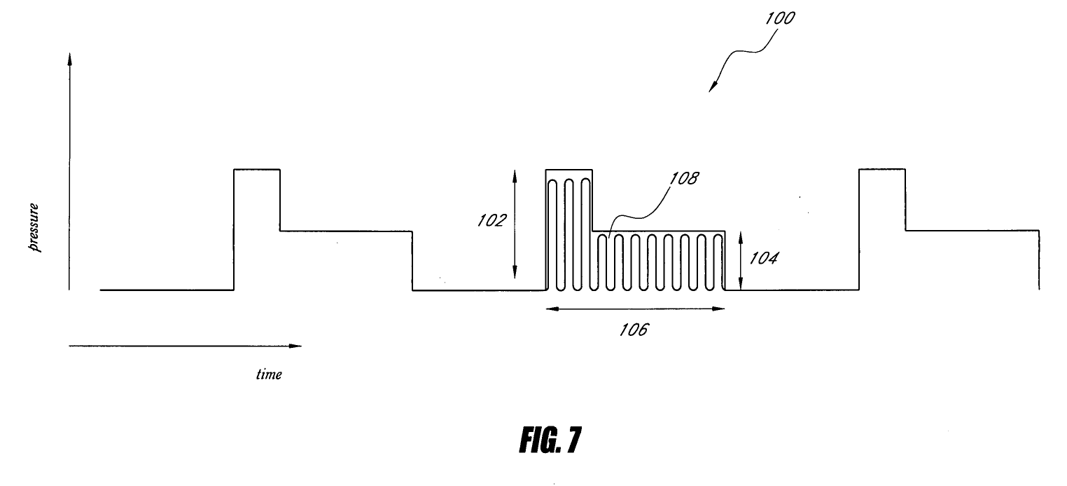

In other embodiments, the amount of cavitation generated at a treatment site is measured by observing bubble activity using a ultrasound imaging system, such as a SONOSITE® 180 portable ultrasound imaging system, available from SonoSite, Inc. (Bothell, Wash.). In such embodiments, the amount of bubble activity is quantifiable by assigning a value 1 to time periods wherein bubble activity is observed, and assigning a value 0 to time periods wherein bubble activity is not observed. The average of these binary scores corresponds to the probability that bubbles are produced for a given configuration. FIGS. 5A and 5B are sonograms that illustrate the microbubble activity that is generated when a sanded polyimide tube is positioned in a plasma clot and is exposed to ultrasonic energy with a peak acoustic pressure of 5.1 MPa. In embodiments wherein the pulse profile of the ultrasonic energy includes multiple pressure amplitudes, such as illustrated in FIG. 7, cavitation activity is optionally measured separately during the high pressure amplitude and the low pressure amplitude phases of the ultrasonic energy pulses.

When a catheter that includes a cavitation promoting surface is positioned within a vascular occlusion, the amount of cavitation generated upon application of ultrasonic energy is enhanced by also supplying a therapeutic compound to the vascular occlusion. For example, FIG. 5A illustrates the microbubble activity when no therapeutic compound is added to the plasma clot, while FIG. 5B illustrates a significant increase in microbubble activity when 1.0 mL of therapeutic compound is added to the plasma clot. Without being limited by theory, this effect is believed to result from the therapeutic compound “softening”, “opening” or partially lysing the occlusion in the region of the cavitation promoting surface, thereby allowing bubbles to be more easily produced in the surrounding fluid environment.

In an example embodiment, an ultrasound catheter is used to expose a plasma clot to ultrasonic energy and a therapeutic compound for approximately 30 minutes. The pulse duration is approximately 50 burst cycles at a pulse repetition frequency of about 1 Hz, which corresponds to a duty cycle of approximately 0.003%. This produces an acoustic spatial average pressure of about 2.4 MPa, and a spatial peak pressure of approximately 2.8 MPa at the outer surface of the ultrasound catheter. In embodiments wherein the ultrasound catheter includes a cavitation promoting surface, lysis of the plasma clot is enhanced by approximately 15.6%±5.83% compared to embodiments wherein the ultrasound catheter does not include a cavitation promoting surface. Thus, the ultrasound-based thrombolysis procedure is enhanced by using a cavitation promoting surface to increase the amount of cavitation at the treatment site. In some embodiments, use of a cavitation promoting surface allows enhanced lysis to be achieved notwithstanding a reduction in the amount of ultrasonic energy delivered to the treatment site.

As described herein, and as illustrated in FIG. 4, certain roughened and/or hydrophobic surfaces provide nucleation sites for free microbubbles, thereby enabling cavitation to be enhanced when the surface is exposed to ultrasonic energy. Hydrophobic surfaces are also used in certain embodiments to increase catheter lubricity, thereby facilitating delivery of the catheter to an intravascular treatment site. Polyimide is a relatively hydrophobic material that is biocompatible and commonly used in the manufacture of intravascular catheters. In certain embodiments, the hydrophobicity of polyimide is increased by application of highly hydrophobic coatings such as silicon-based and polytetrafluoroethylene-based compounds. In other embodiments, the hydrophobicity of polyimide is increased by compounding or blending pre-dispersed hydrophobic particles into the polyimide.

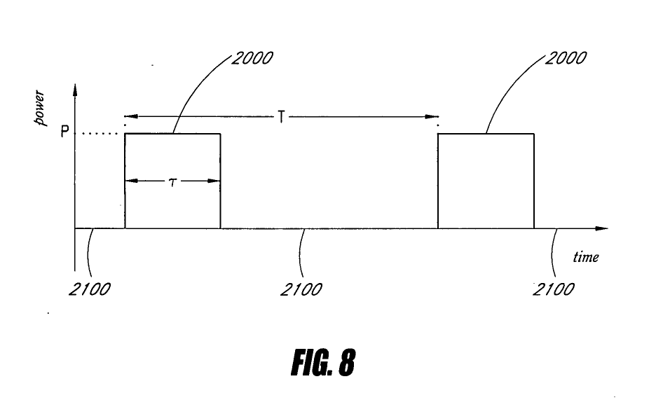

For example, polytetrafluoroethylene is a particle that can be blended into polyimide and that has other significant advantages, such as a relatively low kinetic coefficient of friction (μₖ) compared to other polymers, and a static coefficient of friction (μₛ) that is lower than its kinetic coefficient of friction (μₖ). The size and concentration of the blended polytetrafluoroethylene particles influences the texture and hydrophobicity of the resulting cavitation promoting surface. FIG. 6A is a microscopic image (200×) of a plain polyimide surface, while FIG. 6B is a microscopic image (200×) of a polyimide surface having polytetrafluoroethylene particles dispersed therein.

In other embodiments, a cavitation promoting surface is obtained by roughening a catheter surface. In one such embodiment, roughening is accomplished by sanding using a micro-abrasion equipment and an abrasive having a grid size that is selected based on the level of roughness to be obtained. For example, one suitable abrasive is a powder of aluminum oxide particles having an average diameter of approximately 25 μm. Aluminum oxide and other similar abrasives are dry media, which advantageously facilitate cleaning of the catheter surface after the roughening treatment is performed. In other embodiments, water-based or grease-based compounds are used to make finer abrasions in the catheter surface that would otherwise be possible using dry abrasion media. Use of water-based compounds advantageously facilitates cleaning of the catheter surface after treatment, as compared to grease-based compounds. Water-based and grease-based compounds are compatible with both manual application techniques and machine-based application techniques. For example, one suitable application technique involves immersing the catheter in an abrasion compound and agitating the compound using ultrasonic energy, thereby causing the fine particles in the compound to scrub against the catheter body and produce scratches and crevices therein. In one embodiment, the catheter surface is not so rough that the surface becomes thrombogenic and promotes clot formation when in contact with blood.

In an example embodiment, lysis of a vascular occlusion is accomplished by the delivery of ultrasonic energy from a catheter with a cavitation promoting surface. For instance, in one embodiment the ultrasonic energy has a duty cycle that is preferably between about 0.001% and about 0.005%, and that is more preferably about 0.003%. In another embodiment, the ultrasonic energy has a duty cycle that is preferably between about 3.5% and about 13.5%, and that is more preferably about 8.5%. The ultrasonic energy has a frequency that is preferably between about 1.2 MHz and about 2.2 MHz, and is more preferably about 1.7 MHz. The ultrasonic energy has a pulse repetition frequency that is preferably between about 0.5 Hz and about 1.5 Hz, and that is more preferably about 1 Hz. The ultrasonic energy has a pulse duration that preferably includes between about 5000 burst cycles and about 7000 burst cycles, and that more preferably includes about 5950 burst cycles. The ultrasonic energy has a peak acoustic pressure that is preferably between about 1.8 MPa and about 3.8 MPa, and that is more preferably about 2.8 MPa. The ultrasonic energy has a spatial average acoustic pressure that is preferably between about 1.4 MPa and about 3.4 MPa, and that is more preferably about 2.4 MPa. However, in modified embodiments higher peak acoustic pressure are generated without causing substantial transducer damage by making appropriate adjustments to the frequency, duty cycle and/or pulse duration of the ultrasonic energy.

As described herein, it is possible to damage the treatment site if excess ultrasonic energy is delivered to the patient's vasculature. For example, such damage can be caused by excess thermal energy or excess shear stresses generated by the ultrasonic energy. Additionally, overheating and functioning at high pressure amplitude can substantially reduce the operating lifetime of the ultrasound radiating member. Thus, in an example embodiment the ultrasound catheter is operated in a way that reduces the likelihood of damaging the treatment site and/or the ultrasound radiating member. One way of accomplishing this is to reduce the amount of time the ultrasound member is delivering ultrasonic energy, which subsequently leads to a reduction in the average power delivered to the treatment site. Another way of accomplishing this is to position a cavitation promoting surface at the treatment site.

For example, in certain embodiments an ultrasound radiating member is operated in a pulsed mode, such as by using modulated electrical drive power instead of continuous electrical drive power. In such embodiments, the duty cycle is chosen to avoid causing thermal damage to the treatment site and/or to the ultrasound radiating member. The beneficial effect of the ultrasonic energy does not cease immediately when the ultrasonic energy is switched off. Thus, in certain embodiments the amplitude of the ultrasonic energy and/or the duration of ultrasonic energy delivery is increased to provide a greater clinical effect, while the duty cycle of the ultrasonic energy is reduced to avoid causing thermal damage.

In certain configurations the beneficial effect of ultrasonic energy is maintained notwithstanding a subsequent decrease in ultrasonic power delivered to the treatment site. For example, in certain applications the presence of ultrasound-induced cavitation at the treatment site causes a beneficial effect. Typically ultrasonic energy having a power greater than a cavitation threshold power Cₜ must be delivered to the treatment site to induce cavitation. However, to maintain the cavitation at the treatment site a reduced amount of power Cₘ must be delivered to the treatment site, wherein Cₘ<Cₜ. Therefore, in such embodiments an initial pulse of power Cₜ is delivered to the treatment site to induce cavitation, after which a reduced amount of power Cₘ is delivered to the treatment site to maintain cavitation.

FIG. 8 illustrates an example ultrasonic waveform. In certain applications, such a waveform provides a therapeutic effect when delivered to a treatment site in a patient's vasculature, optionally in conjunction with the delivery of a therapeutic compound. As illustrated, the waveform includes a series of pulses 2000 of ultrasonic energy having peak power P and duration τ. The pulses 2000 are separated by “off” periods 2100. The cycle period T is defined as the time between pulse initiations, and thus the pulse repetition frequency (“PRF”) is given by T⁻¹. The duty cycle is defined as the ratio of time of one pulse to the time between pulse initiations τT⁻¹, and represents the fraction of time that ultrasonic energy is being delivered to the treatment site. The average power delivered in each cycle period is given by PτT⁻¹.

In one example embodiment wherein ultrasonic energy is used to enhance the effect of a therapeutic compound delivered to an intravascular treatment site, the peak power P is between approximately 5 watts and approximately 25 watts. The duty cycle is preferably greater than approximately 0.04, is more preferably greater than approximately 0.06, and is most preferably greater than approximately 0.085. The average power is greater than or equal to approximately 0.45 watts and the pulse repetition frequency is approximately 30 Hz. The pressure generated by such a waveform is preferably greater than about 1 MPa, more preferably greater than about 2 MPa, and most preferably greater than about 2.5 MPa.

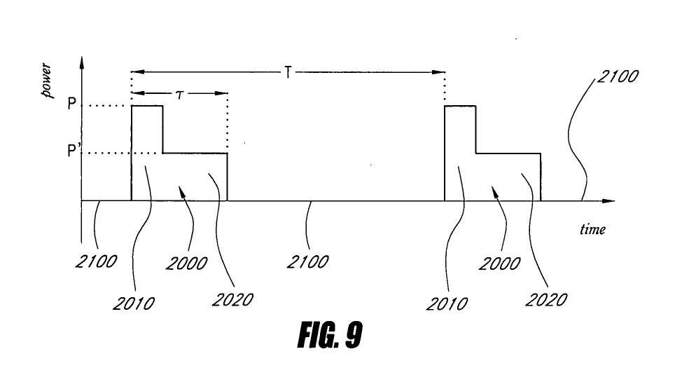

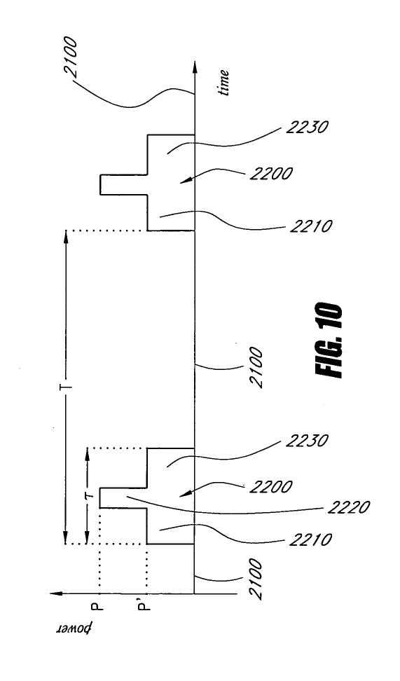

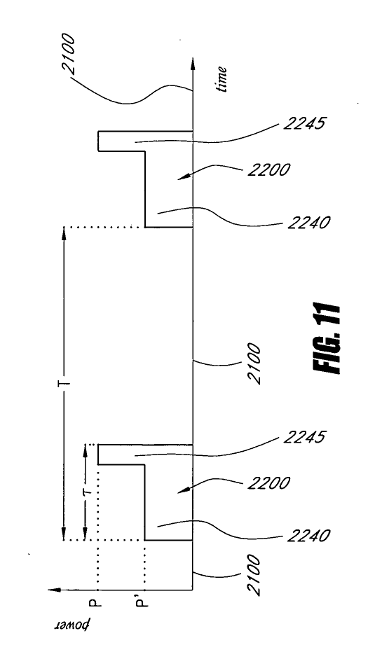

In a modified embodiment, a reduced average power is delivered to the treatment site without significantly reducing the beneficial effect of the ultrasonic energy. Delivering a reduced average power also advantageously reduces the likelihood of causing thermal damage to the treatment site and/or the ultrasound radiating member. FIG. 9 illustrates a modified ultrasonic waveform having a reduced average power as compared to the example waveform illustrated in FIG. 8. The modified ultrasonic waveform illustrated in FIG. 9 is also useful for providing a therapeutic effect when delivered to a treatment site in a patient's vasculature.

The modified ultrasonic waveform illustrated in FIG. 9 comprises a series of pulses 2000 of ultrasonic energy having a peak power P during a first pulse portion 2010, and a reduced power P′ during a second pulse portion 2020. In one application, the waveforms illustrated in FIGS. 8 and 9 have the same cycle period T and pulse duration τ. In another application, the waveform illustrated in FIG. 9 has an increased duty cycle as compared to the waveform illustrated in FIG. 8. In either case, the waveform illustrated in FIG. 9 has a reduced average power as compared to the waveform illustrated in FIG. 8 because the peak power P is not delivered during the entire pulse duration τ. However, the waveform illustrated in FIG. 9 is still useful for providing a therapeutic effect when delivered to a treatment site in a patient's vasculature. For example, in one embodiment the peak power P is of sufficient magnitude to induce cavitation at the treatment site, while the reduced power P′ is of sufficient magnitude to maintain cavitation at the treatment site.

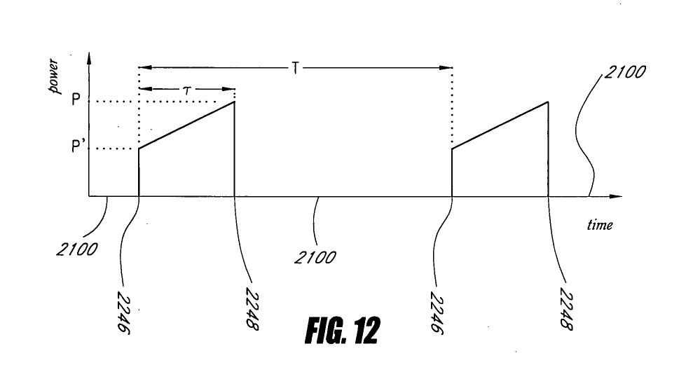

FIG. 10 illustrates another modified ultrasonic waveform having a reduced average power as compared to the example waveform illustrated in FIG. 8. The modified ultrasonic waveform illustrated in FIG. 10 is also useful for providing an enhanced therapeutic effect when delivered to a treatment site in a patient's vasculature. Such a waveform comprises a series of pulses 2200 of ultrasonic energy having a reduced power P′ during a beginning pulse portion 2210 and an ending pulse portion 2230, and a peak power P during an intermediate pulse portion 2220. The power during the beginning pulse portion 2210 and the ending pulse portion 2230 is not required to be equal. The waveforms illustrated in FIGS. 8 and 10 have the same cycle period T and pulse duration τ. The modified ultrasonic waveform illustrated in FIG. 10 has a reduced average power as compared to the waveform illustrated in FIG. 8 because the peak power P is not delivered during the entire pulse duration τ. However, the waveform illustrated in FIG. 10 is still useful for providing a therapeutic effect when delivered to a treatment site in a patient's vasculature.

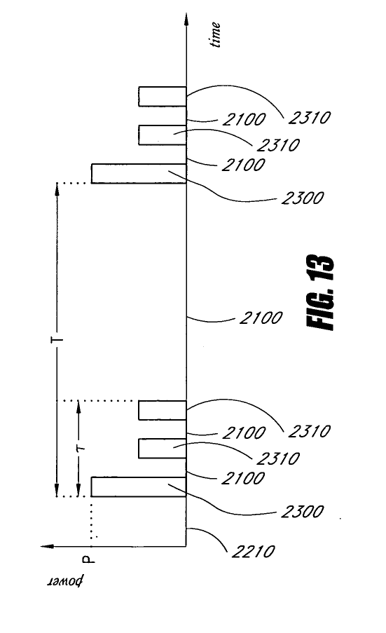

FIG. 11 illustrates another modified ultrasonic waveform having a reduced average power as compared to the example waveform illustrated in FIG. 8. The modified ultrasonic waveform illustrated in FIG. 11 is also useful for providing a therapeutic effect when delivered to a treatment site in a patient's vasculature. Such a waveform comprises a series of pulses 2200 of ultrasonic energy having a reduced power P′ during a first pulse portion 2240, and a peak power P during a second pulse portiori 2245. The waveforms illustrated in FIGS. 8 and 11 have the same cycle period T and pulse duration τ. The modified ultrasonic waveform illustrated in FIG. 11 has a reduced average power as compared to the waveform illustrated in FIG. 8 because the peak power P is not delivered during the entire pulse duration τ. However, the waveform illustrated in FIG. 11 is still useful for providing a therapeutic effect when delivered to a treatment site in a patient's vasculature.

FIG. 12 illustrates another modified ultrasonic waveform having a reduced average power as compared to the example waveform illustrated in FIG. 8. The modified ultrasonic waveform illustrated in FIG. 12 is also useful for providing a therapeutic effect when delivered to a treatment site in a patient's vasculature. Such a waveform comprises a series of pulses 2200 of ultrasonic energy that have a reduced power P′ at a beginning pulse portion 2246, and that have a gradually increasing power until a peak power P is generated at an ending pulse portion 2248. The waveforms illustrated in FIGS. 8 and 12 have the same cycle period T and pulse duration τ. The modified ultrasonic waveform illustrated in FIG. 12 has a reduced average power as compared to the waveform illustrated in FIG. 8 because the peak power P is not delivered during the entire pulse duration τ. However, the waveform illustrated in FIG. 12 is still useful for providing a therapeutic effect when delivered to a treatment site in a patient's vasculature.

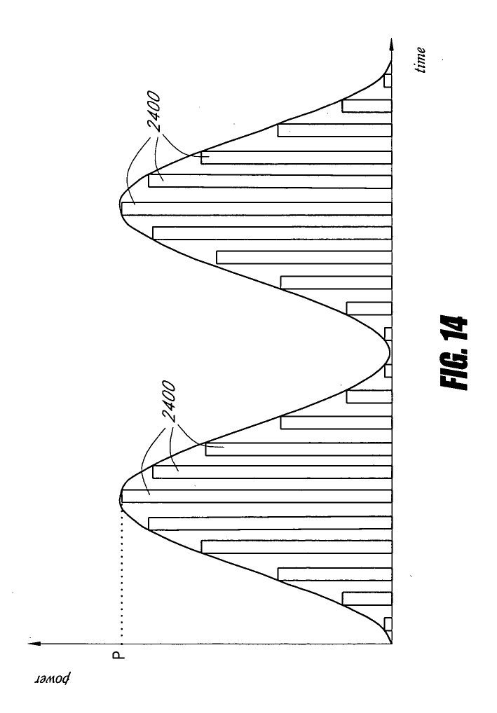

FIG. 13 illustrates another modified ultrasonic waveform having a reduced average power as compared to the example waveform illustrated in FIG. 8. The modified ultrasonic waveform illustrated in FIG. 13 is also useful for providing a therapeutic effect when delivered to a treatment site in a patient's vasculature. Such a waveform comprises a high amplitude pulse 2300 having a peak power P, and one or more low amplitude pulses 2310 having a reduced power. While FIG. 13 illustrates that the high amplitude pulse 2300 is delivered before the one or more low amplitude pulses 2310, other delivery sequences are used in other embodiments. For example, in one embodiment at least one of the low amplitude pulses is delivered before the high amplitude pulse 2300. The waveforms illustrated in FIGS. 8 and 13 have the same cycle period T and pulse duration τ. The modified ultrasonic waveform illustrated in FIG. 13 has a reduced average power as compared to the waveform illustrated in FIG. 8 because the peak power P is not delivered during the entire pulse duration τ. However, the waveform illustrated in FIG. 13 is still useful for providing a therapeutic effect when delivered to a treatment site in a patient's vasculature.

In a modified embodiment, the amplitude of the waveform illustrated in FIG. 13 is adjusted such that the average power is increased as compared to the example waveform illustrated in FIG. 8. In such embodiments, one or more high amplitude pulses 2300 are delivered to the patient's vasculature, followed by one or more reduced amplitude pulses 2310. For example, in one application the high amplitude pulses 2300 have a peak power P that is approximately equal to the peak power that can be reliably delivered from the ultrasound radiating member without damaging the ultrasound radiating member. Such an embodiment is optionally used in conjunction with a cavitation promoting surface, as described herein.

For instance, in one embodiment between about 3 and about 100 burst cycles of ultrasonic energy having a peak power P of greater than or equal to about 20 watts, and creating a peak pressure of greater than about 2.5 MPa, are delivered to the treatment site. These high amplitude pulses 2300 are followed by a plurality of reduced amplitude pulses 2310 having a power that is between approximately 7 watts and approximately 8 watts. The number of reduced amplitude burst cycles that are delivered to the treatment site is preferably between about 5000 and about 10000, and is more preferably between about 6500 and about 7500. This configuration results in delivery to the treatment site of ultrasonic energy having average power of greater than about 0.45 watts at a duty cycle of greater than about 0.085.

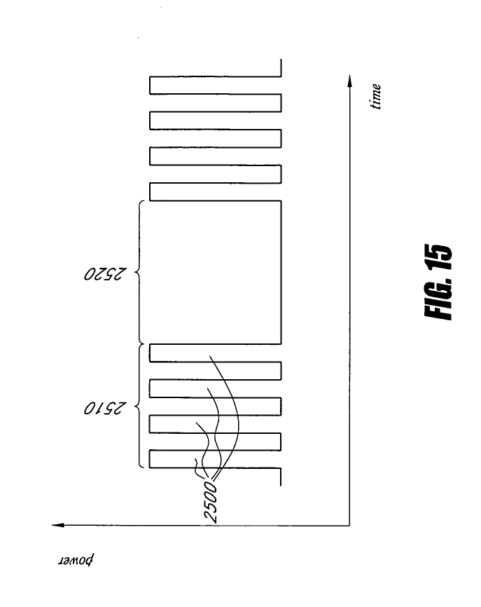

FIG. 14 illustrates another modified ultrasonic waveform having a reduced average power as compared to the example waveform illustrated in FIG. 8. The modified ultrasonic waveform illustrated in FIG. 14 is also useful for providing a therapeutic effect when delivered to a treatment site in a patient's vasculature. Such a waveform comprises a sequence of pulses 2400 that have a sinusoidally-varying power. In one embodiment, certain of the pulses 2400 have a power that is greater than the peak power P of the waveform illustrated in FIG. 8. However, in such embodiments, the modified ultrasonic waveform illustrated in FIG. 14 still has a reduced average power as compared to the waveform illustrated in FIG. 8 because the peak power P is delivered for a relatively short time period as compared to the cycle period T. The waveform illustrated in FIG. 14 is particularly useful for a therapeutic effect when delivered to a treatment site in a patient's vasculature because it is capable of simultaneously providing both high power pulses of ultrasonic energy and a reduced average power delivery.

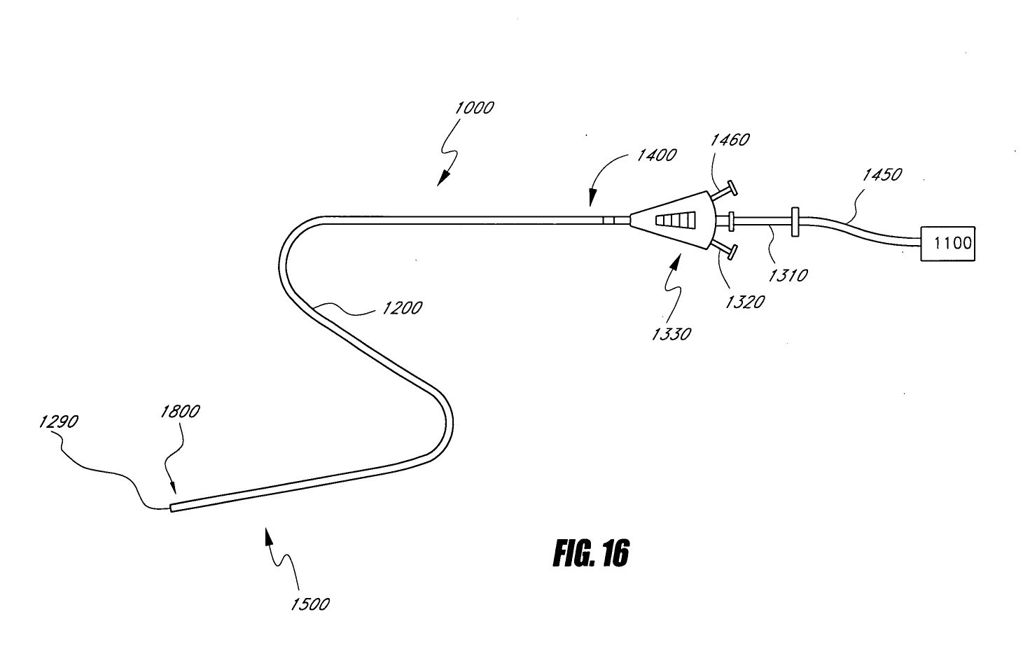

FIG. 15 illustrates another modified ultrasonic waveform having a reduced average power as compared to the example waveform illustrated in FIG. 8. The modified ultrasonic waveform illustrated in FIG. 15 is also useful for providing a therapeutic effect when delivered to a treatment site in a patient's vasculature. Such a waveform comprises a plurality of pulses 2500 that are delivered in an envelope 2510 that is followed by a period 2520 in which little or no ultrasonic energy is delivered. In one embodiment, the pulses 2500 delivered in envelope 2510 have a peak power that is greater than the peak power P of the waveform illustrated in FIG. 8. However, in such embodiments, the modified ultrasonic waveform illustrated in FIG. 15 still has a reduced average power as compared to the waveform illustrated in FIG. 8 because the aggregate duration of the pulses 2500 illustrated in FIG. 15 is significantly less than the pulse duration τof the waveform illustrated in FIG. 8. This is accomplished by virtue of the fact that ultrasonic energy is not continuously delivered for the duration of the envelope 2510.

In one embodiment, the duration of envelope 2510 is greater than or equal to the duration of the period 2520. In another embodiment, the duration of envelope 2510 is less than the duration of the period 2520. Although four pulses are illustrated as being delivered during the envelope 2510 in FIG. 15, more or fewer pulses are delivered in other embodiments. The waveform illustrated in FIG. 15 is particularly useful for a therapeutic effect when delivered to a treatment site in a patient's vasculature because it is capable of simultaneously providing both high power pulses of ultrasonic energy and a reduced average power delivery.

In certain embodiments wherein the ultrasound radiating member is a PZT transducer, the PZT transducer is excited by specific electrical parameters that cause it to vibrate in a way that generates ultrasonic energy. Suitable vibration frequencies for the ultrasound radiating member include, but are not limited to, from about 20 kHz to less than about 20 MHz. In one embodiment, the vibration frequency is between about 500 kHz and about 20 MHz, and in another embodiment the vibration frequency is between about 1 MHz and about 3 MHz. In yet another embodiment, the vibration frequency is about 3 MHz. Within these frequency ranges, the in vivo production of cavitation and/or enhancement of the effect of a therapeutic compound is optionally improved by using particular electrical parameters to produce one or more of the waveforms disclosed herein.

In one example embodiment, the average power delivered in each cycle period is preferably between about 0.1 watts and about 2.0 watts, is more preferably between about 0.5 watts and about 1.5 watts, and is most preferably between about 0.6 watts and about 1.2 watts. In one example embodiment, the duty cycle is preferably between about 1% and about 50%, is more preferably between about 5% and about 25%, and is most preferably between about 7.5% and about 15%. In one example embodiment, the peak power P delivered to the treatment site is preferably between about 0.1 watts and about 20 watts, is more preferably between about 5 watts and about 20 watts, and is most preferably between about8 watts and about 16 watts. The pulse amplitude during each pulse is constant or varied. Other parameters are used in other embodiments depending on the particular application.

The effect of a therapeutic compound is optionally enhanced by using a certain pulse repetition frequency PRF and/or a certain pulse duration τ. In one example embodiment, the PRF is preferably between about 5 Hz and about 150 Hz, is more preferably between about 10 Hz and about 50 Hz, and is most preferably between about 20 Hz and about 40 Hz. In one example embodiment, the pulse duration τ is preferably between about 1 millisecond and about 50 milliseconds, is more preferably between about 1 millisecond and about 25 milliseconds, and is most preferably between about 2.5 milliseconds and about 5 milliseconds.

In one example embodiment, the ultrasound radiating member used with the electrical parameters described herein operates with an acoustic efficiency that is preferably greater than about 50%, that is more preferably greater than about 75%. The ultrasound radiating member is formed using a variety of shapes, such as, for example, a solid cylinder, a hollow cylinder, a flat polygon, a bar-shaped polygon, a triangular-shaped polygon, and the like. In an example embodiment wherein the ultrasound radiating member has an elongate shape, the length of the ultrasound radiating member is between about 0.1 centimeters and about 0.5 centimeters, and the thickness or diameter of the ultrasound radiating member is between about 0.02 centimeters and about 0.2 centimeters.

In one embodiment the duty cycle is manipulated based on a temperature reading taken at the treatment site during delivery of ultrasonic energy. As described herein, in certain embodiments a temperature sensor is positioned at the treatment site to measure the temperature at the treatment site during delivery of ultrasonic energy. The temperature at the treatment is optionally monitored to detect whether a threshold temperature is exceeded. For example, in one embodiment, the threshold temperature is set based on a temperature at which there is an increased danger of causing thermal damage to the patient's vasculature. In certain embodiments, if the threshold temperature is exceeded, one or more of the operating characteristics of the ultrasound energy is modified to reduce the average power of ultrasonic energy delivered to the treatment site. In another embodiment, the threshold temperature is set based on a temperature at which there is an increased danger of causing thermal damage to the ultrasound radiating member, for example by significantly reducing the operating lifetime of the ultrasound radiating member.

For example, in one embodiment, the duty cycle is increased if the threshold temperature is exceeded. In an example embodiment wherein the duty cycle is increased if the threshold temperature is exceeded, the duty cycle is increased by an interval that is preferably between about 0.01 and 0.50, that is more preferably between about 0.05 and 0.25, that is even more preferably between about 0.05 and 0.15, and that is most preferably between about 0.06 and 0.10.

In other embodiments, one or more other operating characteristics of the ultrasonic energy is adjusted if the threshold temperature is exceeded; examples of such characteristics include peak power P, average power, and pulse repetition frequency PRF. In still other embodiments, delivery of ultrasonic energy is paused if the threshold temperature is exceeded, thereby providing a cooling period for the treatment site and/or the ultrasound radiating member to return to a reduced temperature. In one embodiment, the duration of the cooling period at least partially depends on a temperature measured at the treatment site during the cooling period.

Although some of the embodiments disclosed herein are described in the context of a PZT transducer, certain features and aspects are applicable to an ultrasound radiating member that is not a PZT transducer. That is, in certain embodiments operating the ultrasound radiating member using pulsed waveforms, or modulated electrical drive power instead of continuous drive power, has utility outside the context of a PZT transducer. Certain of the embodiments disclosed herein enhance clinical effects of a therapeutic compound while reducing the likelihood of causing thermal damager to the treatment site and/or the ultrasound radiating member.

Furthermore, certain of the embodiments disclosed herein are compatible with ultrasound catheters having a plurality of ultrasound radiating members positioned therein. In one such embodiment, a first one of the plurality of ultrasound radiating members is driven using a first waveform, and a second one of the plurality of ultrasound radiating members is driven using a second waveform that is different from the first waveform. Likewise, in another such embodiment, a first group of the plurality of ultrasound radiating members is driven using a first waveform, and a second group of the plurality of ultrasound radiating members is driven using a second waveform. Thus, in certain embodiments ultrasonic energy having more than one waveform is delivered to the patient's vasculature, optionally simultaneously.

Additionally, the ultrasound waveforms disclosed herein are optionally used in conjunction with a cavitation promoting surface that is positioned at the treatment site. As disclosed herein, a cavitation promoting surface advantageously reduces the acoustic pressure amplitude required to initiate cavitation at the treatment site, thus allowing the parameters of the ultrasonic energy to be optionally adjusted. For example, in certain embodiments use of a cavitation promoting surface enables the parameters of the ultrasonic energy to be adjusted so as to reduce the amount of thermal or mechanical stress generated at the treatment site, or inflicted on the ultrasound radiating member itself.

Under certain conditions, the acoustic pressures used to initiate cavitation causes thermal damage to the treatment site and/or substantially reduce the operating lifetime of the ultrasound radiating member. In such embodiments, this is addressed by initially driving the ultrasound radiating member using a modified acoustic pulse profile, as illustrated in FIG. 7. For example, in one embodiment the ultrasound radiating member is initially driven at an increased first pressure amplitude 102 to nucleate microbubbles and initiate cavitation, and is subsequently driven at a reduced second pressure amplitude 104 to maintain the efficacy of the of the treatment without causing substantial damage to the treatment site and/or substantially reducing the operating lifetime of the ultrasound radiating member. In certain embodiments, the reduced second pressure amplitude is sufficient to activate microbubbles nucleated using ultrasonic energy having the first pressure amplitude. The pulse profile 100 is also useful in embodiments wherein ultrasonic energy provided with first pressure amplitude 102 results in reduced lysis as compared to ultrasonic energy provided with the second pressure amplitude 104. Optionally, the relative amplitude and duration of the first and second pressure amplitudes are manipulated to influence whether stable or inertial cavitation is generated after the microbubble nucleation phase.

### SCOPE OF THE INVENTION

While the foregoing detailed description discloses several embodiments of the present invention, it should be understood that this disclosure is illustrative only and is not limiting of the present invention. It should be appreciated that the specific configurations and operations disclosed can differ from those described above, and that the methods described herein can be used in contexts other than treatment of vascular occlusions.

I claim:

1. A method of applying ultrasonic energy to a treatment site within a patient's vasculature, the method comprising:
positioning an ultrasound radiating member at a treatment site within a patient's vasculature; and
activating the ultrasound radiating member to produce pulses of ultrasonic energy at a cycle period T≦1 second, wherein each pulse of ultrasonic energy has a first peak amplitude for a first duration, and a second reduced amplitude that is less than the first peak amplitude for a second duration.

2. The method of claim 1, further comprising positioning a cavitation promoting surface at the treatment site, such that the cavitation promoting surface is present at the treatment site when the ultrasound radiating member is activated.

3. The method of claim 1, wherein at least a portion of the second duration occurs before the first duration is initiated.

4. The method of claim 1, wherein the ultrasound radiating member is movable with respect to a catheter sheath that is positioned at the treatment site.

5. The method of claim 1, further comprising delivering a therapeutic compound to the treatment site concurrently with the ultrasonic energy.

6. The method of claim 1, wherein the ultrasound radiating member operates with an acoustic efficiency greater than about 50%.

7. The method of claim 1, wherein the first peak amplitude induces cavitation at the treatment site.

8. The method of claim 1, wherein the first duration is shorter than the second duration.

9. The method of claim 1, wherein the pulses of ultrasonic energy have a duty cycle that is between about 1% and about 50%.

10. The method of claim 9, further comprising:
measuring a temperature at the treatment site; and
adjusting the duty cycle based on the temperature measurement.

11. A method comprising:
positioning an ultrasound radiating member at a treatment site within a patient's vasculature;
delivering pulses of ultrasonic energy to the treatment site from the ultrasound radiating member, wherein the pulses of ultrasonic energy include a variable amplitude, such that the pulses have an increased pulse amplitude during a first pulse segment, and a reduced pulse amplitude during a second pulse segment; and
delivering a therapeutic compound to the treatment site simultaneously with the delivery of the pulses of ultrasonic energy.

12. The method of claim 11, wherein the first pulse segment occurs before the second pulse segment.

13. The method of claim 11, wherein the second pulse segment occurs before the first pulse segment.

14. The method of claim 11, wherein the pulses have a pulse amplitude that varies linearly between the increased pulse amplitude and the reduced pulse amplitude.

15. The method of claim 11, wherein the pulses have a cycle period T≦1 second.

16. The method of claim 15, wherein the sum of a duration of the first pulse segment and a duration of the second pulse segment is between about 5% and about 25% of the cycle period T.

17. The method of claim 11, wherein:
a plurality of ultrasound radiating members are positioned at the treatment site;
a first ultrasonic waveform is delivered from a first ultrasound radiating member to the treatment site; and
a second ultrasonic waveform is delivered from a second ultrasound radiating member to the treatment site.

18. The method of claim 17, wherein the first ultrasonic waveform and the second ultrasonic waveform are delivered to the treatment site simultaneously.

19. A method comprising:
positioning a catheter at a treatment site within a patient's vasculature, the catheter being positioned at least partially within an occlusion at the treatment site;
delivering a therapeutic compound from the catheter to the occlusion; and
delivering a plurality of packets ultrasonic energy from an ultrasound radiating member positioned within the catheter to the occlusion, wherein the packets of ultrasonic energy comprise a plurality of pulses of ultrasonic energy having an amplitude that varies pulse-to-pulse.

20. The method of claim 19, wherein the catheter includes a cavitation promoting surface that is exposed to the packets of ultrasonic energy

21. The method of claim 19, wherein the packets of ultrasonic energy are temporally separated by a period wherein substantially no ultrasonic energy is delivered to the treatment site.

22. The method of claim 19, wherein the plurality of pulses of ultrasonic energy have an amplitude that varies sinusoidally from pulse-to-pulse.

23. The method of claim 19, wherein the plurality of pulses of ultrasonic energy includes at least one trigger pulse having sufficient power to induce cavitation at the treatment site.

24. The method of claim 19, further comprising measuring a temperature at the treatment site after at least one of the packets of ultrasonic energy is delievered to the occlusion.

25. The method of claim 24, further comprising modifying the amplitude of the plurality of pulses of ultrasonic energy in response to the temperature measurement.

26. The method of claim 19, wherein the ultrasound radiating member is movable with respect to the catheter.

27. An ultrasound catheter configured to be inserted into a patient's vascular system, the catheter comprising:
an elongate outer sheath defining a central lumen that extends longitudinally from an outer sheath proximal region to an outer sheath distal region;
an elongate hollow inner core positioned in the central lumen, the inner core defining a utility lumen; and
a ultrasound radiating member having a hollow inner passage through which the inner core passes, wherein the ultrasound radiating member is positioned generally between the inner core and the outer sheath;
wherein the outer sheath includes an outer surface, the outer sheath outer surface having
a cavitation promoting region located adjacent to the ultrasound radiating member, and
a smooth region located proximal to the cavitation promotion region, wherein the cavitation promoting region has an increased surface roughness as compared to the smooth region.

28. The ultrasound catheter of claim 27, wherein the elongate outer sheath has an outer diameter of less than about 5.2 French.

29. A catheter system for delivering ultrasonic energy and a therapeutic compound to a treatment site within a body lumen, the catheter system comprising:
a tubular body having
a proximal end,
a distal end, and
an energy delivery section positioned between the proximal end and the distal end, wherein the energy delivery section includes a cavitation promoting surface having an increased surface roughness;
a fluid delivery lumen extending at least partially through the tubular body and having at least one outlet in the energy delivery section;
an inner core configured for insertion into the tubular body, the inner core comprising a plurality of ultrasound radiating members connected to an elongate electrical conductor; and
wiring such that a voltage can be applied from the elongate electrical conductor across a selected plurality of the ultrasound radiating members, such that the selected plurality of ultrasound radiating members can be driven simultaneously.

30. A method of treating a vascular occlusion, the method comprising:
delivering a catheter with a plurality of ultrasound radiating members to a treatment site within a patient's vasculature, wherein:
the vascular occlusion is located at the treatment site and
the catheter includes a cavitation promoting surface region having an increased surface roughness as compared to surface regions adjacent the cavitation promoting surface region; and
delivering ultrasonic energy to the treatment site from the catheter so as to generate cavitation at the treatment site.

31. The method of claim 30, further comprising delivering an ultrasound contrast agent to the treatment site.

32. The method of claim 30, wherein the ultrasonic energy has a duty cycle that is between about 1% and about 10%.

33. The method of claim 30, wherein the ultrasonic energy has a frequency that is between about 1.2 MHz and about 2.2 MHz.

34. The method of claim 30, wherein the ultrasonic energy has a peak acoustic pressure that is between about 1.8 MPa and about 3.8 MPa.

35. The method of claim 30, wherein the ultrasonic energy has a spatial average acoustic pressure that is preferably between about 1.4 MPa and about 3.4 MPa.

36. An ultrasound catheter comprising:
an elongate tubular body having a proximal region and a distal region, wherein an energy delivery section is included within the distal region of the tubular body;
an ultrasound radiating member positioned adjacent to the energy delivery section of the elongate tubular body;
a cavitation promoting surface that is formed on an exterior surface of the ultrasound catheter, and that is exposed to ultrasonic energy when the ultrasound radiating member is activated;
a fluid delivery lumen positioned within the elongate tubular body; and
a fluid delivery port that is configured to deliver a fluid within the fluid delivery lumen to an exterior region of the ultrasound catheter that is adjacent to the cavitation promoting surface.

37. The ultrasound catheter of claim 36, wherein the fluid delivery lumen passes through a hollow inner core of the ultrasound radiating member.

38. The ultrasound catheter of claim 36, wherein the fluid delivery port is positioned at a distal end of the elongate tubular body.

39. The ultrasound catheter of claim 36, wherein the fluid delivery port is positioned on the exterior surface of the ultrasound catheter.

40. The ultrasound catheter of claim 36, wherein the fluid delivery port is positioned on the cavitation promoting surface.

41. The ultrasound catheter of claim 36, wherein when the ultrasound radiating member is activated, cavitation occurs adjacent to the cavitation promoting surface, but does not occur adjacent to other regions of the catheter.

42. The ultrasound catheter of claim 36, wherein the cavitation promoting surface is configured to entrap gas pockets thereon when immersed in a liquid.

43. A catheter system comprising:
an elongate tubular body having a distal region and a proximal region opposite the distal region;
an ultrasound radiating member positioned adjacent to the distal region of the elongate tubular body;
a fluid delivery lumen extending through at least a portion of the elongate tubular body;
a fluid delivery port that is configured to deliver a fluid within the fluid delivery lumen to a region exterior to the elongate tubular body; and
a control system configured to provide a control signal to the ultrasound radiating member, wherein the control signal causes the ultrasound radiating member to generate a plurality of pulses of ultrasonic energy, and wherein a first pulse of ultrasonic energy has an amplitude that is greater than a second pulse of ultrasonic energy.

44. The catheter system of claim 43, further comprising a cavitation promoting surface that is exposed to ultrasonic energy when the control signal is provided to the ultrasound radiating member.

45. The catheter system of claim 44, wherein the control signal is configured to cause cavitation in a region adjacent to the cavitation promoting surface, but to not cause cavitation adjacent to other regions of the catheter.

46. The catheter system of claim 44, wherein the cavitation promoting surface is configured to entrap gas pockets thereon when immersed in a liquid.

47. The catheter system of claim 43, wherein the plurality of pulses of ultrasonic energy have an amplitude that varies sinusoidally from pulse-to-pulse.

48. The catheter system of claim 43, wherein the first pulse of ultrasonic energy has a peak power of greater than about 15 watts.

49. The catheter system of claim 43, further comprising a temperature sensor, wherein the control system is configured to modify the control signal based on a temperature signal generated by the temperature sensor.

50. A catheter system comprising:
an elongate tubular body having a distal region and a proximal region opposite the distal region;
an ultrasound radiating member positioned adjacent to the distal region of the elongate tubular body;
a fluid delivery lumen extending through at least a portion of the elongate tubular body;
a fluid delivery port that is configured to deliver a fluid within the fluid delivery lumen to a region exterior to the elongate tubular body; and
a control system configured to provide a control signal to the ultrasound radiating member, wherein the control signal causes the ultrasound radiating member to generate pulses of ultrasonic energy at a cycle period T≦1 second, wherein a selected pulse of ultrasonic energy has
a first peak amplitude for a first duration, and a second reduced amplitude that is less than the first peak amplitude for a second duration.

51. The catheter system of claim 50, further comprising a cavitation promoting surface that is exposed to ultrasonic energy when the control signal is provided to the ultrasound radiating member.

52. The catheter system of claim 51, wherein the control signal is configured to cause cavitation in a region adjacent to the cavitation promoting surface, but to not cause cavitation adjacent to other regions of the catheter.

53. The catheter system of claim 51, wherein the cavitation promoting surface is configured to entrap gas pockets thereon when immersed in a liquid.

54. The catheter system of claim 50, wherein at least a portion of the second duration occurs before the first duration is initiated.

55. The catheter system of claim 43, wherein the first peak amplitude has a peak power of greater than about 15 watts.

56. The catheter system of claim 43, further comprising a temperature sensor, wherein the control system is configured to modify the control signal based on a temperature signal generated by the temperature sensor.
